(19) United States
(12) Patent Application Publication
(10) Pub. No.: US 2015/0045730 A1
(43) Pub. Date: Feb. 12, 2015

(54) PNEUMATIC RETRACTABLE SELF-DESTRUCTING INJECTION DEVICE

(71) Applicant: Genshun CAI, Zhejiang (CN)

(72) Inventor: Cai Genshun, Zhejiang (CN)

(21) Appl. No.: 14/384,786

(22) PCT Filed: Apr. 27, 2013
(86) PCT No.: PCT/CN2013/074838

§ 371 Date: Sep. 12, 2014

(30) Foreign Application Priority Data
Jun. 30, 2012 (CN) 201210221311.1

(51) Int. Cl. A61M 5/32; A61M 5/50

(52) U.S. Cl. CPC A61M 5/3221; A61M 5/50; A61M 2005/3231
USPC 604/110

(57) ABSTRACT

A pneumatic retractable auto-disable syringe, comprising a syringe barrel (4), a hollow cylindrical plunger (3), a needle hub (5), a sealing ring (6), a piston (8) and a seal connector (7), wherein: the seal connector (7) and the needle hub (5) are provided with a compatible connection mechanism disposed thereon; the inner wall of the syringe barrel (4) at the front end thereof and the outer wall of the needle hub (5) at the front end thereof are pneumatically sealed; the plunger (3) is provided with a position limiting device (31) disposed on the inner wall thereof; the plunger (3) is provided with an air vent (33) disposed at the bottom end thereof. The present invention is structurally simple, easy and inexpensive to manufacture, and easily assembled. The auto-disablement thereof is divided into two steps: first, the seal connector (7) is inserted into a hole in the needle hub (5) by means of manual force, and the seal connector (7) becomes loose once the injection fluid is completely injected; second, as the sealing ring (6) continues to be pushed forward, air pressure is generated and the seal connector (7) is then ejected into the plunger (3) by the energy released from the compressed air. The lower precision requirements of the product allow for simpler and more reliable auto-disablement.

### FIELD

The present invention falls within the field of syringe technology and specifically relates to a pneumatic retractable auto-disable syringe.

### BACKGROUND

Chinese Patent ZL200920113284.X discloses an auto-disable safety vacuum syringe comprising a syringe barrel and a plunger wherein a needle hub with a needle mounted thereon is disposed at the front end of the plunger, characterized in that: the needle hub is provided with a through hole connecting the needle and the syringe barrel; the plunger is a hollow cylindrical plunger with a vacuum push rod inserted thereinto; a seal connector is mounted at the front end of the plunger; the plunger forms a vacuum cavity between the seal connector and the vacuum push rod; the seal connector and the needle hub are provided with a connection mechanism disposed therebetween for connecting the seal connector and the needle hub when the plunger is pushed to the front end of the syringe barrel; the vacuum push rod and the syringe barrel are provided with a position limiting mechanism disposed therebetween for axially positioning the vacuum push rod when the plunger is pushed to a set position.

The plunger is pushed forward during an injection and once the injection fluid is completely injected, the plunger will reach the front end of the syringe barrel. At this point, the seal connector located at the front end of the plunger and the needle hub mounted with a needle are connected through the connection mechanism, the position limiting mechanism causes the vacuum push rod to be fixed instead of moving forward along with the plunger, and since the air pressure inside the plunger decreases, the seal connector and the needle hub that are connected together are sucked into the plunger due to atmospheric pressure outside.

The aforesaid technique has the following disadvantages: 1) the syringe has many fittings, the structures of its vacuum push rod and plunger are relatively complicated, and the cost is relatively high; 2) assembly of the syringe is complicated, and this is especially so for assembly of the plunger thereof when it is necessary to first push the seal connector from the rear portion of the plunger to the front end of the plunger by means of an ejector pin, to put the vacuum push rod into the plunger, to push the vacuum push rod from the rear portion of the plunger to the front end of the plunger in a predetermined manner, to pull the vacuum push rod to a specific distance before rotating the vacuum push rod for connecting the bottom end of the vacuum push rod to the position limiting mechanism; and due to the complicated assembly processes thereof, the special assembly machinery designed has a poor reliability and its mass production is difficult; as a result, all the assembly processes have to be handled manually and this generates heavy workload; 3) this relatively complicated technique is difficult to implement and associated with high cost, poor market acceptance level, difficulties in promoting, and poor practicality.

### SUMMARY OF THE INVENTION

To overcome the aforesaid deficiencies of the current technique, the present invention is directed to provide a pneumatic retractable auto-disable syringe that is structurally simple, safe and reliable, less costly, highly practical and easily accepted by market.

To this end, the present invention adopts the following technical scheme:

A pneumatic retractable auto-disable syringe, comprising a syringe barrel, a hollow cylindrical plunger, a needle hub, a sealing ring, a piston and a seal connector, wherein: the needle hub is provided with a needle centrally disposed thereon, the needle hub is mounted at the front end of the syringe barrel after passing through the sealing ring located therebeneath, the piston is disposed on the outer side of the plunger at the front end thereof, the seal connector is disposed on the inner side of the plunger at the front end thereof, the seal connector and the needle hub are provided with a compatible connection mechanism disposed thereon, characterized in that: when the plunger is pushed to the front end of the syringe barrel, the inner wall of the syringe barrel at the front end thereof and the outer wall of the needle hub at the front end thereof are pneumatically sealed; the plunger is provided with a position limiting device disposed at the bottom portion thereof for limiting the range of movement of the seal connector and the needle hub in the plunger after the auto-disablement thereof; the plunger is provided with an air vent disposed at the bottom end thereof for air exchange between the inner cavity of the plunger and the outside.

Preferably, the bottom portion of the needle hub is outwardly bulged to form a position limiting pedestal, the sealing ring is mounted onto the position limiting pedestal and the outer wall of the needle hub and the inner wall of the syringe barrel are pneumatically sealed, and the internal diameter of the sealing ring is larger than the external diameter of the needle hub located above the position limiting pedestal.

Preferably, the quantity of the position limiting devices is two, and they are disposed respectively at the middle and bottom portions thereof; the position limiting devices each comprise three convex ribs disposed on the inner wall of the plunger.

Preferably, the air vent comprises a plurality of aeration holes disposed on the side wall of the plunger at the bottom portion thereof.

Preferably, the specific structure of the pneumatic seal between the inner wall of the syringe barrel at the front end thereof and the outer wall of the needle hub at the front end thereof is as follows: the front end of the syringe barrel is radially inwardly shrunk to form a retention slot and the inner wall of the retention slot is provided with a bevel; the front end of the needle hub is radially inwardly shrunk to form a step complete with a transitional bevel thereon, and the bevel on the step matches the bevel on the retention slot.

Preferably, the position limiting device is a narrow mouth formed by radial inward contraction at the bottom portion of the plunger; a smooth transitional bevel is disposed between the upper side of the narrow mouth and the inner wall of the plunger; and the narrow mouth and the seal connector have a slight interference fit.

Preferably, the position limiting device is a bottom cover embedded in the plunger at the bottom portion thereof.

The present invention has the following major advantages compared to the current technique.

1. The present invention has relatively less fittings, the front end of the syringe barrel and the front end of the needle hub are pneumatically sealed, the bottom end of the needle hub and the inner wall of the syringe barrel are pneumatically sealed by means of the sealing ring; thus the plunger requires no vacuum push rod and its structural simplicity contributes to easy processing and low manufacturing cost.

2. Assembly of the present invention is simple. The plunger can be assembled by directly pushing the seal connector from the rear portion of the plunger to the end of the plunger by means of the ejector pin; this mode is easy to operate and assembly can be performed by mechanical means, thus resulting in lower labor intensity.

3. The front end of the syringe barrel of the present invention is radially inwardly shrunk to form a retention slot and the inner wall of the retention slot is provided with a bevel; the front end of the needle hub is radially inwardly shrunk to form a step complete with a transitional bevel thereon, and the bevel on the step matches the bevel on the retention slot. If a slight gap exists between the front end of the needle hub and the inner wall of the syringe barrel at the front end thereof during assembly, once the plunger is pushed to the front end of the syringe barrel, the seal connector first comes into contact with the needle hub causing it to be subjected to an upward force and tightly pressed against the end of the syringe barrel, thereby enabling a reliable pneumatic seal between the front end of the syringe barrel and the front end of the needle hub.

4. The auto-disablement of the pneumatic retractable auto-disable syringe of the present invention is divided into two steps: first, when the plunger is pushed to the front end of the syringe barrel, the seal connector first comes into contact with and is connected to the needle hub, a gap still exists between the front end of the plunger and the underside of the sealing ring, and the plunger is pushed to loosen the seal connector and the plunger; second, the plunger is pushed forward to have the sealing ring separated from the needle hub, so that it is possible for the auto-disablement of the syringe to take place with a relatively small force applied by medical personnel and for the extent of air compression required for auto-disablement to be reduced, thereby enabling easier and more reliable auto-disablement of the syringe.

### BRIEF DESCRIPTION OF THE DRAWINGS

FIG. 1 is a schematic diagram illustrating the present invention.

FIG. 2 is an enlarged view at A of FIG. 1.

FIG. 3 is an exploded view of the present invention.

FIG. 4 is a schematic diagram of the present invention post auto-disablement.

FIG. 5 is a sectional view B-B of the syringe barrel shown in FIG. 3.

FIG. 6 is a schematic diagram illustrating the pneumatic seal between the front end of the syringe barrel and the front end of the needle hub.

FIG. 7 is a schematic diagram illustrating another embodiment of the present invention.

FIG. 8 is an enlarged view at C of FIG. 7.

### DETAILED DESCRIPTION

### Embodiment 1

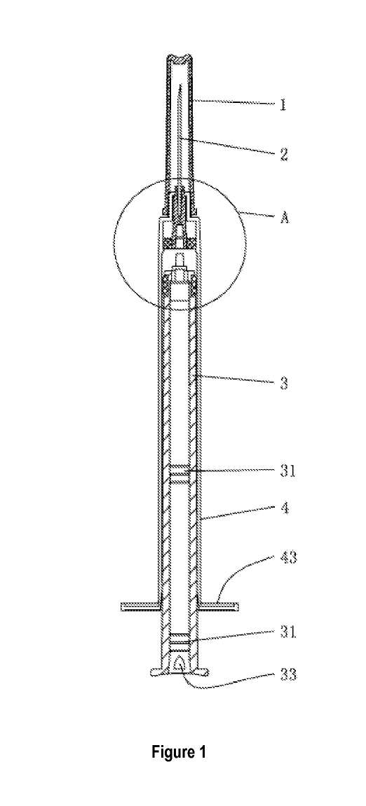

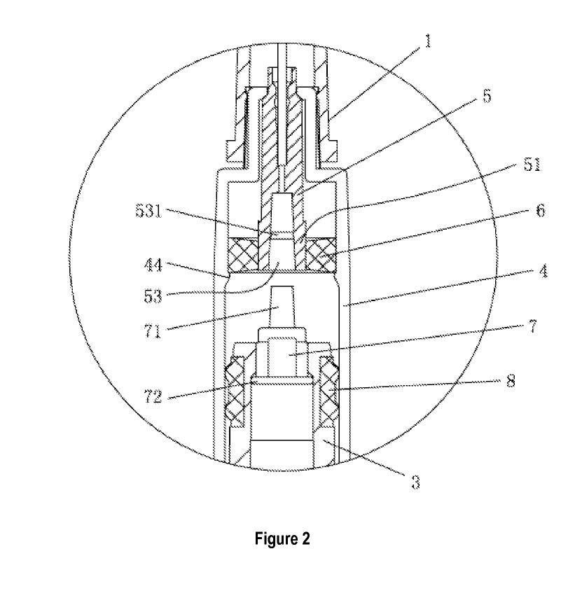

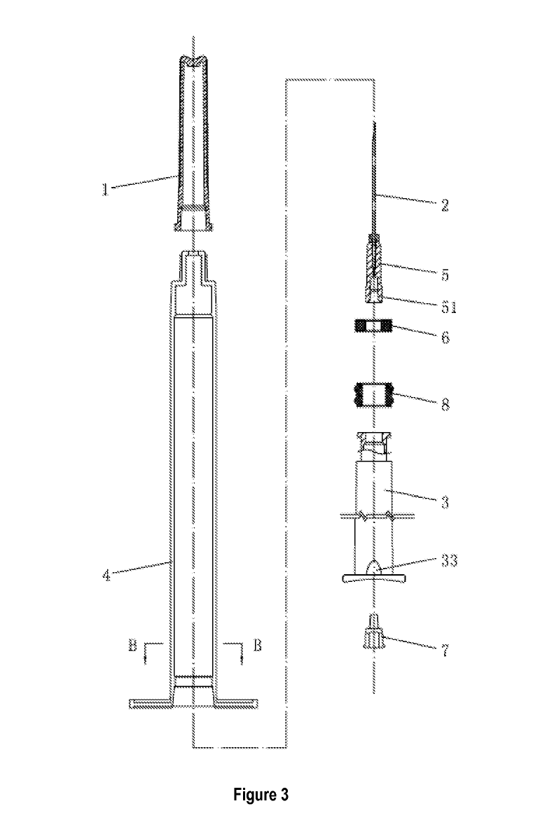

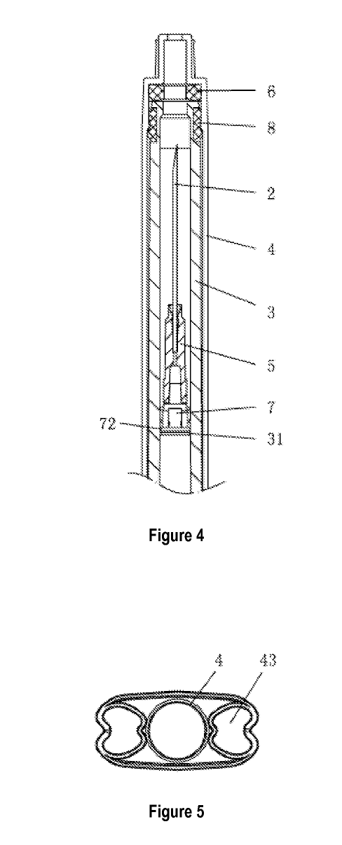

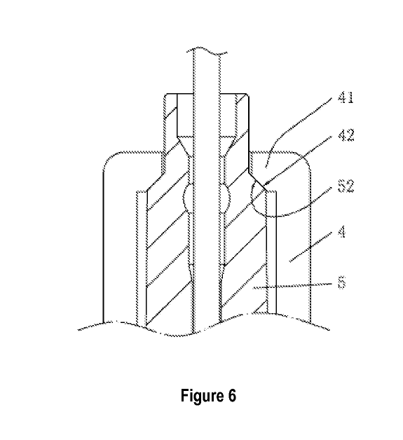

FIGS. 1˜6 illustrate a pneumatic retractable auto-disable syringe comprising a syringe barrel 4, a hollow cylindrical plunger 3, a needle hub 5, a sealing ring 6, a piston 8 and a seal connector 7, wherein: the needle hub 5 is provided with a needle 2 centrally disposed thereon, the needle hub 5 is mounted at the front end of the syringe barrel 4 after passing through the sealing ring 6 located therebeneath, the piston 5 is disposed on the outer side of the plunger 3 at the front end thereof, the seal connector 7 is disposed on the inner side of the plunger 3 at the front end thereof, the seal connector 7 and the needle hub 5 are provided with a compatible connection mechanism disposed thereon, characterized in that: when the plunger 3 is pushed to the front end of the syringe barrel 4, the inner wall of the syringe barrel 4 at the front end thereof and the outer wall of the needle hub 5 at the front end thereof are pneumatically sealed; the plunger 3 is provided with a position limiting device 31 disposed on the inner wall thereof for preventing the seal connector 7 and the needle hub 5 from being ejected from the bottom end of the plunger 3 by the energy released from the compressed air; the plunger 3 is provided with an air vent 33 disposed at the bottom end thereof for air exchange between the inner cavity of the plunger 3 and the outside; the air vent 33 comprises a plurality of aeration holes disposed on the side wall of the plunger 3 at the bottom portion thereof.

In this embodiment, since the seal connector 7 is pushed from the bottom end of the plunger 3 to the neck of the plunger 3 when being assembled, medical personnel's finger presses against the through hole at the bottom end of the plunger 3 (the bottom edge of the plunger's inner cavity). In other words, the inner cavity of the plunger 3 is not connected with the outside during injection and auto-disablement; it is therefore necessary to have aeration holes disposed at the bottom portion of the plunger 3 for air exchange with the outside.

The bottom portion of the needle hub 5 is outwardly bulged to form a position limiting pedestal 51, the sealing ring 6 is mounted onto the position limiting pedestal 51 and the outer wall of the needle hub 5 and the inner wall of the syringe barrel 4 are pneumatically sealed, and the internal diameter of the sealing ring 6 is larger than the external diameter of the needle hub 5 located above the position limiting pedestal 51.

The quantity of the position limiting devices 31 is two, and they are disposed respectively at the middle and bottom portions of the plunger 3; the position limiting devices 31 each comprise three convex ribs disposed on the inner wall of the plunger 3 with the three convex ribs forming two recessed grooves. The flange 72 at the bottom portion of the seal connector 7 is embedded into one of the recessed grooves under inertia effect. The position limiting device 31 located at the bottom portion of the plunger 3 is primarily for safety purposes. The flange 72 of the seal connector 7 and the position limiting device 31 work in coordination to prevent residual injection fluid from moving backwards and to prevent the needle 2 from protruding from the plunger 3.

The specific structure of the pneumatic seal between the inner wall of the syringe barrel 4 at the front end thereof and the outer wall of the needle hub 5 at the front end thereof is as follows: the front end of the syringe barrel 4 is radially inwardly shrunk to form a retention slot 41 and the inner wall of the retention slot 41 is provided with a bevel 42; the front end of the needle hub 5 is radially inwardly shrunk to form a step complete with a transitional bevel 52 thereon, and the bevel 52 on the step and the bevel 42 on the retention slot 41 have an identical degree of inclination, and the degree of inclination is within the range of 0˜60°. In this embodiment, the degree of inclination is 45° which should be a preferred choice.

The needle hub 5 is provided with an recessed cavity 53 disposed at the bottom portion thereof, the recessed cavity 53 is an inwardly tapered conical recessed cavity, the front end of the seal connector 7 is a forwardly tapered conical plug 71, and the plug 71 is provided with a circulation channel disposed at the side face thereof. When the plug 71 and the needle hub 5 begin to connect with each other, a small amount of injection fluid still exists between the needle hub 5 and the plunger 3, and the small amount of injection fluid cay enter the needle hub 5 through the circulation channel. The perfect matching between the tapers and sizes of the recessed cavity 53 and the plug 71 helps to minimize the amount of residual injection fluid in the needle hub 5. Upon completion of the connection between the plug 71 and the needle hub 5, the injection fluid is completely injected. The recessed cavity 53 is provided with an annular ridge 531 disposed on the inner wall thereof, and the annular ridge 531 and the plug 71 work in coordination to enable the seal connector 7 to lead the needle hub 5 back into the plunger 3 with the help of pneumatic energy. The external diameter of the middle portion of the seal connector 7 matches the inner wall of the plunger 3 at the end portion thereof, so that the injection fluid will not permeate into the plunger 3 under a certain pressure.

The finger-supported portion 43 of the syringe barrel 4 is provided with an apple-shaped or heart-shaped reinforcement rib.

The syringe barrel 4 is provided with a position limiting bulge 44 disposed on the inner wall at the front portion thereof and matching the sealing ring 6. The position limiting bulge 44 is capable of preventing the sealing ring 6 and the needle hub 5 from being caused by external factors to move downward, thereby ensuring the pneumatic seal between the front end of the needle hub 5 and the inner wall of the syringe barrel 4 at the front end thereof. The working mechanism of the present invention is as follows: when the plunger 3 is pushed to the front end of the syringe barrel 4, the seal connector 7 first comes into contact with the needle hub 5, and after the seal connector 7 is connected to the needle hub 5, a gap still exists between the front end of the plunger 3 and the underside of the sealing ring 6. As the plunger 3 continues to be pushed forward causing the front end of the plunger 3 to press against the bottom side of the sealing ring 6, the connection between the seal connector 7 and the plunger 3 has been loosened by the needle hub 5. The internal diameter of the sealing ring 6 is larger than the external diameter of the needle hub 5 located above the position limiting pedestal 51. The plunger 3 continues to be pushed forward to have the sealing ring 6 separated from the needle hub 5. At this point, pneumatic energy is released from the compressed air at the front portion of the syringe barrel 4 to push the needle hub 5 and the seal connector 7 into the inner cavity of the plunger 3, and the flange 72 of the seal connector 7 is embedded into the position limiting device 31 located on the inner wall of the plunger 3 under inertia effect.

A slight gap may exist between the front end of the needle hub 5 and the inner wall of the syringe barrel 4 at the front end thereof (the needle hub 5 is not precisely pushed to the desired position) during assembly due to staff negligence, in which case when the plunger 3 is pushed to the front end of the syringe barrel 4, the seal connector 7 first comes into contact with the needle hub 5 causing it to be subjected to an upward force and tightly pressed against the end of the syringe barrel 4, thereby maintaining a pneumatic seal between the front end of the syringe barrel 4 and the inner wall at the front end of the needle hub 5 during the auto-disablement process.

### Embodiment 2

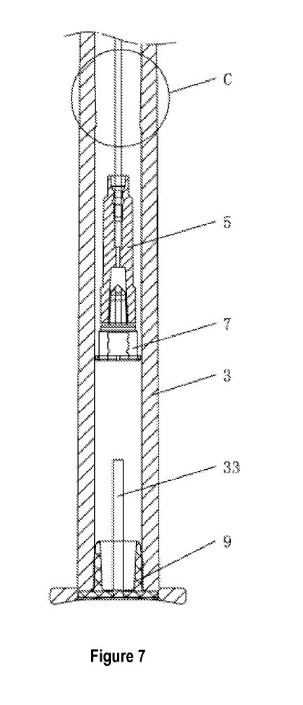

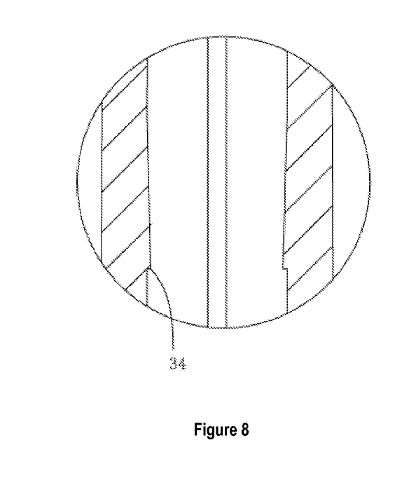

As shown in FIGS. 7˜8, this embodiment is basically identical to Embodiment 1. The differences between the two are: the position limiting device on the plunger 3 is a narrow mouth 34 formed on the inner wall thereof, and a transitional bevel is disposed between the upper side of the narrow mouth 34 and the inner wall of the plunger 3; a bottom cover 9 is embedded in the plunger at the bottom portion thereof, and the air vent 33 for air exchange between the inner cavity of the plunger 3 and the outside is bar-shaped. After auto-disablement of the syringe, the seal connector 7 and the needle hub 5 are located between the narrow mouth 34 and the bottom cover 9.

Finally, it must be noted that the foregoing embodiments are only intended to illustrate the technical scheme of the present invention and not to limit the scope thereof. Notwithstanding the detailed description in connection with the preferred embodiments, it should be apparent to those of ordinary skill in the art that various modifications or substitutions that may be made to the embodiments without departing from the spirit and scope of the present invention shall fall within the scope of the appended claims.

1. A pneumatic retractable auto-disable syringe, comprising a syringe barrel, a hollow cylindrical plunger, a needle hub, a sealing ring, a piston and a seal connector, wherein: the needle hub is provided with a needle centrally disposed thereon, the needle hub is mounted at the front end of the syringe barrel after passing through the sealing ring located therebeneath, the piston is disposed on the outer side of the plunger at the front end thereof, the seal connector is disposed on the inner side of the plunger at the front end thereof, the seal connector and the needle hub are provided with a compatible connection mechanism disposed thereon, characterized in that: when the plunger is pushed to the front end of the syringe barrel, the inner wall of the syringe barrel at the front end thereof and the outer wall of the needle hub at the front end thereof are pneumatically sealed; the plunger is provided with a position limiting device disposed at the bottom portion thereof for limiting the range of movement of the seal connector and the needle hub in the plunger after the auto-disablement thereof; the plunger is provided with an air vent disposed at the bottom end thereof for air exchange between the inner cavity of the plunger and the outside.

2. A pneumatic retractable auto-disable syringe as defined in claim 1, characterized in that the bottom portion of the needle hub is outwardly bulged to form a position limiting pedestal, the sealing ring is mounted onto the position limiting pedestal and the outer wall of the needle hub and the inner wall of the syringe barrel are pneumatically sealed, and the internal diameter of the sealing ring is larger than the external diameter of the needle hub located above the position limiting pedestal.

3. A pneumatic retractable auto-disable syringe as defined in claim 1, characterized in that the quantity of the position limiting devices is two, and they are disposed respectively at the middle and bottom portions thereof; the position limiting devices each comprise three convex ribs disposed on the inner wall of the plunger.

4. A pneumatic retractable auto-disable syringe as defined in claim 1, characterized in that the air vent comprises a plurality of aeration holes disposed on the side wall of the plunger at the bottom portion thereof.

5. A pneumatic retractable auto-disable syringe as defined in any of claims 1 through 4, characterized in that the specific structure of the pneumatic seal between the inner wall of the syringe barrel at the front end thereof and the outer wall of the needle hub at the front end thereof is as follows: the front end of the syringe barrel is radially inwardly shrunk to form a retention slot and the inner wall of the retention slot is provided with a bevel; the front end of the needle hub is radially inwardly shrunk to form a step complete with a transitional bevel thereon, and the bevel on the step matches the bevel on the retention slot.

6. A pneumatic retractable auto-disable syringe as defined in claim 1, characterized in that the position limiting device is a narrow mouth formed by radial inward contraction at the bottom portion of the plunger; a smooth transitional bevel is disposed between the upper side of the narrow mouth and the inner wall of the plunger; and the narrow mouth and the seal connector have a slight interference fit.

7. A pneumatic retractable auto-disable syringe as defined in claim 1, characterized in that the position limiting device is a bottom cover embedded in the plunger at the bottom portion thereof.
